(19) United States
(12) Patent Application Publication
(10) Pub. No.: US 2017/0340276 A1
(43) Pub. Date: Nov. 30, 2017

(54) SHOE WITH INTEGRALLY CONNECTED SLEEVE

(71) Applicants: AVA NOORZAI, CAMARILLO, CA (US); SOPHIA NOORZAI, CAMARILLO, CA (US); OLIVIA NOORZAI, CAMARILLO, CA (US); HANA NOORZAI, CAMARILLO, CA (US); JENNIFER NOORZAI, CAMARILLO, CA (US); MUSTAFA NOORZAI, CAMARILLO, CA (US)

(72) Inventors: AVA NOORZAI, CAMARILLO, CA (US); SOPHIA NOORZAI, CAMARILLO, CA (US); OLIVIA NOORZAI, CAMARILLO, CA (US); HANA NOORZAI, CAMARILLO, CA (US); JENNIFER NOORZAI, CAMARILLO, CA (US); MUSTAFA NOORZAI, CAMARILLO, CA (US)

(21) Appl. No.: 15/603,965

(22) Filed: May 24, 2017

Related U.S. Application Data
(60) Provisional application No. 62/340,544, filed on May 24, 2016.

(51) Int. Cl. A61B 5/00; A41D 17/00; A41D 31/00; A43B 3/00; A63B 71/12; A61F 5/01; A61B 5/11; A61B 5/0205; A41D 1/00; A43B 5/10; A43B 5/06; A43B 5/02; A43B 5/00; A43B 5/12; A61B 5/024

(52) U.S. Cl. CPC A61B 5/6802; A43B 5/12; A41D 17/005; A41D 31/0088; A43B 3/0005; A63B 71/1225; A61F 5/0116; A61F 5/0109; A61F 5/0104; A61B 5/11; A61B 5/0205; A41D 1/002; A43B 5/10; A43B 5/06; A43B 5/02; A43B 5/002; A43B 5/001; A61B 5/024; A41D 2400/26; A63B 2071/125; A63B 2071/1258

(57) ABSTRACT

An athletic shoe with a legging sleeve connected thereto. The athletic shoe may take any of a variety of high-performance shoes known in the art, such as tennis shoes, basketball shoes, and the like. The legging sleeve component is integrally connected as part of the shoe and is extensible over the calf, knees and thigh of the wearer and operative to form a compressive fit thereabout. Integrated as part of such legging sleeve may include pockets, channels and pouches for receiving brace structures, support devices, protective coverings and a variety of other implements to facilitate movement, support and promote proper form when playing sports. Such device may further include integration with sensors so as to monitor physical activity, or measurement of a variety of other physiological parameters for use in a variety of applications.

### CROSS-REFERENCE TO RELATED APPLICATIONS

The present patent application claims priority to U.S. Provisional Patent Application Ser. No. 62/340,544, entitled A SHOE WITH AN INTEGRALLY CONNECTING SLEEVE AND/OR BRACE TO HELP IMPROVE SUPPORT, FLEXIBILITY, PROTECTION AND PROVIDE INFORMATION, UV PROTECTION AND BLOOD CIRCULATION TO THE FOOT, ANKLE, LOWER LEG (SHIN), KNEE, UPPER LEG AND THIGH AREA OF THE BODY, filed on May 24, 2016, all of the teachings of which are incorporated herein by reference.

### STATEMENT RE: FEDERALLY SPONSORED RESEARCH/DEVELOPMENT

Not Applicable

### BACKGROUND

The present invention is directed to athletic shoes having a supportive legging sleeve integrated therewith for use in providing enhanced support and protection to the feet, ankle, shin, knee and thigh of the wearer, as well as all muscles, ligaments and tendons associated therewith.

Specially designed athletic footwear to enhance athletic performance is well-known in the art. Exemplary of such footwear includes tennis shoes, basketball shoes, cleats for baseball, soccer and football, plus numerous others. Also well-known are complementary support materials, such as compression legging sleeves, that are operative to form a snug, compressive fitting around foot portions of one or both of the wearer's legs. Such compressive sleeves may take several forms including shin/calf sleeves, knee-sleeves, quad-sleeves and even full length compression leg-sleeves that are operative to extend about the thigh, knee and shin/calf of the wearer.

Exemplary prior art includes all of the following:

U.S. Pat. No. 3,900,898, entitled ATHLETIC SHIELD, issued to Ackerman on Aug. 26, 1975;

U.S. Pat. No. 4,169,324, entitled SOCK AND SHOE AND SOCK AND SHOE FASTENING MEANS, issued to Gibbs et al. on Oct. 2, 1979;

U.S. Pat. No. 4,187,619, entitled STRAP FASTENING MEANS FOR A SOCK AND SHOE COMBINATION, issued to Gibbs et al. on Feb. 12, 1980;

U.S. Pat. No. 6,007,506, entitled METHOD OF USING A SHOE AND SUPPORT DEVICE, issued to Heil on Dec. 28, 1999.

The disclosures of each of the foregoing are incorporated herein by reference.

Presently, however, such products (the footwear and compression legging sleeves) are separate and discrete articles that are not integrated with one another so as to cooperate to promote enhanced support, protection or otherwise facilitate proper athletic form when playing a particular sporting event. This is particularly true of court-based sports, such as tennis, basketball, volleyball and the like, which require the performance of highly specialized movements, extreme agility and quick reflexes.

Also lacking from such disparate articles is the ability to integrate further mechanical structures and supports, such as support braces to protect and restrict the motion of the wearer's feet, ankles, shin, knee and thigh. In such applications, the athlete is seeking to attain an optimal degree of protection for various parts of the wearer's leg while also seeking to be able to perform at the highest athletic ability; however, the athlete must mix and match such components to try to balance, in his or her best judgement, the optimal combination of footwear, bracing/protective wear, and compressive legging. As a consequence, the athlete will often times try to piece together such components, which often times fails and actually hinders the athlete's performance and/or provides a sub-optimal degree of protection or support, whether it be the ankle, knees or other part of the athlete's legs.

Due to these shortcomings, there is a substantial need in the art for a fully integrated combination of athletic footwear with a compressive legging formed integrally therewith that cooperate to provide an optimal degree of compressive support to the feet, ankle and legs of the wearer. There is likewise a need for such a system that can integrate support and protective structures, such as bracing, pads, guards and the like, so as to impart protection to various parts of the wearer's legs, such as the ankles and knees. Still further, there is a need in the art for such a combination that is of simple construction, can be utilized to impart additional desirable features, such as UV protection, easy and extremely comfortable to wear, of low cost to fabricate and can seamlessly integrate and properly orient supportive and protective mechanisms, such as braces for the ankle and/or the knee, shin protection, and the like, that further may be adjustable so as to be selectively deployed from the thigh of the wearer to the wearer's ankle. Additionally desirable is such a footwear/legging combination that provides for integration with electronics so as to measure one or more physiological parameters, such as heart rate and calories burned, distance traveled and the like and can generate data for use in a wide variety of applications, from physical therapy, to training, and even gaming applications, and virtual reality data analysis for gathering physiological data from “smart” sensors with possible MEMS (micro-electromechanical systems) or NENS (nano-electromechanical systems). These wearable smart sensors may use nano microchips with magnetometers, accelerometers, and gyroscopes to measure motion and direction in space. Wearable foot to thigh sensors must withstand the legs normal movements and infringe on the leg as little as possible.

### BRIEF SUMMARY

The present invention specifically addresses and alleviates the above-identified deficiencies in the art. In this regard, the present invention is directed to a combination athletic shoe with a compressive legging sleeve that are operatively interconnected with one another for use in providing support and protection to a wearer's leg extending from the wearer's foot up to the wearer's thigh. The footwear component may comprise any of a variety of conventional athletic or sporting shoes as may be adapted for use in a particular sport. In this regard, it is contemplated that the footwear may take the form of a conventional tennis show, basketball shoe, running shoe and the like, although it is contemplated that virtually any/type of shoe or boot may fall within the scope of the present invention. Along those lines, such footwear may comprise a soccer, baseball or football cleat, golf shoe or spikes, hiking boot or ballet slipper, among others well-known in the art.

With respect to the legging or sleeve component, the same, too, may be of a conventional nature and operative to extend upwardly from the footwear, over the ankle, calf/shin, knee and up to and over the thigh of the wearer when fully extended. To that end, it is contemplated that the legging or sleeve component will be integrally connected with the shoe, either by permanent attachment, or by attachment means such that the sleeve and footwear are at all times maintained as an interconnected unit when worn by the wearer.

To enhance the functionality of the combination footwear with legging sleeve, it is contemplated that the sleeve component may be designed or fabricated in a manner that imparts compressive or selectively compressive support areas anywhere extending from the wearer's ankle and up to the wearer's thigh, as well as any specific anatomical locations in between. Still further, it is contemplated that the sleeve may be fabricated in a manner that enhances visibility, reflection, provides enhanced protection from ultraviolet radiation and/or fabricated in a manner that facilitates ventilation while also providing support, and may further be adorned with decorative indicia, logos and the like.

The legging or sleeve component may further be designed and configured with channels, grooves, pockets, and the like for receiving and interconnecting with protective or support structures, such as bracing for protecting the ankle and/or knees of the wearer, or otherwise be operative to receive, interconnect with and properly position protective shields, guards and the like, such as shin guards or knee pads depending on the intended use of the footwear with compressive sleeve, and the particular protection needs of the wearer. In this regard, it is contemplated that the shoe with integrated sleeve will be specially designed to align with and properly engage such bracing or protective elements and orient the same in an optimal manner so as to achieve the desired degree of protection and/or bracing while at the same time enabling the wearer to have a optimal degree of motion and ability to perform the necessary movements for a given activity.

To further enhance the functionality of the combination athletic shoe with compressive legging, it is contemplated that one or more sensors or other monitoring equipment can be integrated as part of the combination in order to measure a specific physiological parameter, such as heart rate, calories burned or other information such as number of steps taken, distance travelled, elapsed time for a given activity, or any of a variety of parameters or types of data well-known to those skilled in the art. In this regard, it is contemplated that by integrating such sensors and being able to generate and track data related to physical activity can be useful in assessing athletic performance, developing a workout regimen or otherwise integrated as part of a training and/or rehabilitation process. It is further contemplated that such sensors may be operative to both receive and transmit signals, and may impart electrostimulation used in physical therapy applications, as well as training and exercise regimens. The sensors may further be operative to work in a manner that measures and provides guidance to the wearer to perform optimal motions and movements, such as serving a tennis ball, serving a volleyball, performing a specific dance maneuver, perfecting a golf swing, or any of a variety of other activities, including gaming applications, virtual reality analysis and walking.

In order to wear the device, it is presently contemplated that the integrally connected sleeve with shoe will be designed such that the sleeve will have a proximal end through which the wearer will insert his or her foot and thereafter advance the sleeve upwardly towards the leg ultimately to the point where the wearer's foot is retained in the shoe and the sleeve subsequently extending over the ankle, shin/calf and thigh of the wearer. In an alternative embodiment, the sleeve may first be put on by the wearer and then interconnected with the footwear such that the sleeve and footwear are securely interconnected with one another. Other variations for attaching the interconnect footwear with sleeve will also within the skill level of the ordinary artisan.

### BRIEF DESCRIPTION OF THE DRAWINGS

These as well as other features of the present invention will become more apparent upon reference to the drawings.

FIG. 1 is a frontal perspective view of a pair of shoes with integrally connected legging sleeves wherein each shoe and dedicated sleeve attached therewith is constructed in accordance with a preferred embodiment of the present invention.

FIG. 2 is a perspective view of a shoe with integrally connected legging sleeve illustrating in phantom the interconnection between the respective elements.

FIG. 3 is an exploded view of a shoe with legging sleeve operative to be interconnected therewith, as shown in FIG. 2.

FIG. 4 is a perspective view of a shoe and a distal portion of a legging sleeve to be interconnected therewith as shown in accordance with a further embodiment of the present invention.

FIG. 5 is a perspective view of a shoe and a distal portion of a legging sleeve to be interconnected therewith as shown in accordance with a further embodiment of the present invention.

FIG. 6 is a perspective view of a shoe and a distal portion of a legging sleeve to be interconnected therewith as shown in accordance with a further embodiment of the present invention.

FIG. 7 is a shoe with integrally connected sleeve constructed in accordance with a preferred embodiment of the present invention further including a sensor element operatively interconnected with said sleeve component.

### DETAILED DESCRIPTION

The detailed description set forth below is intended as a description of the presently preferred embodiment of the invention, and is not intended to represent the only form in which the present invention may be implemented or performed. The description sets forth the functions and sequences of steps for practicing the invention. It is to be understood, however, that the same or equivalent functions and sequences may be accomplished by different embodiments and that they are also intended to be encompassed within the scope of the invention.

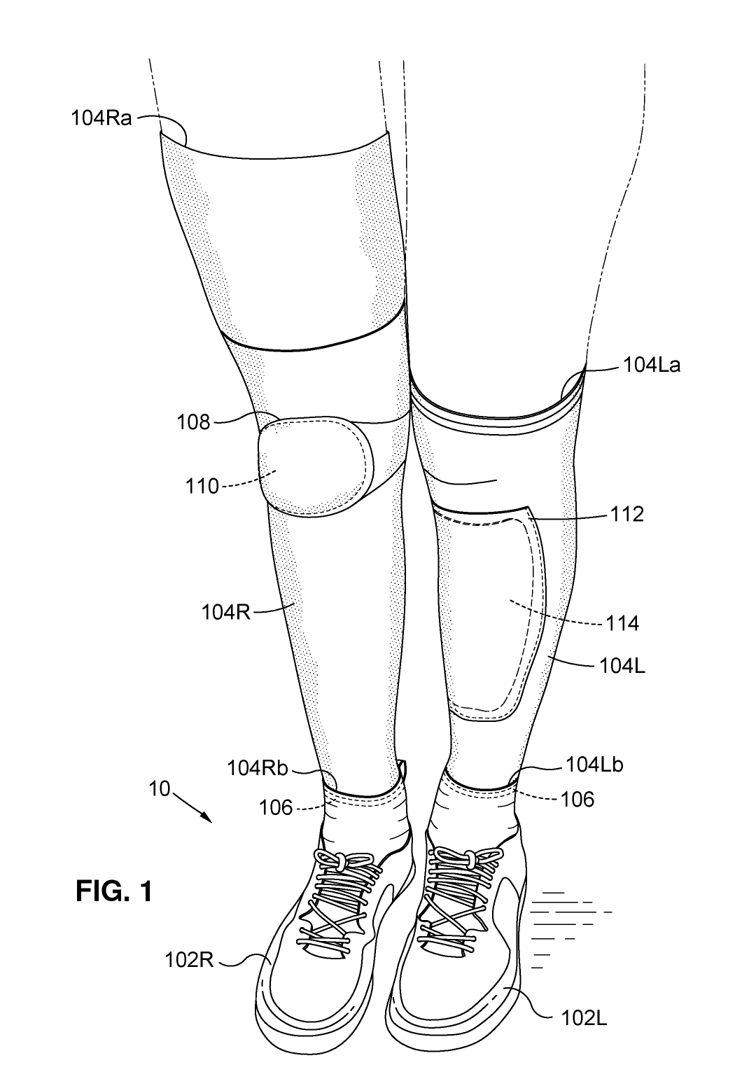

Referring now to the figures, and initially to FIG. 1, there is shown a combination shoe with supportive legging sleeve 10 constructed in accordance with a preferred embodiment of the present invention. As illustrated, the invention 10 will preferably be provided as a matching pair of shoes 102, with a dedicated shoe for the right foot 102R and the left foot 102L. Such shoes 102R, 102L may take any of a variety of shoes known in the art, and in particular athletic shoes intended for a specialized sport or activity. In this regard, it is contemplated that such shoes 102R, 102L may take the form of any of a court-based shoe, such as tennis, basketball, volleyball or the like, as well as running shoes, running spikes for track and field, or other types of cleats and spikes for grass or turf-based sports, such as football cleats, soccer cleats, baseball cleats, golf shoes or spikes, hiking boots and the like. Still further, it is contemplated that shoes 102R, 102L may take the form of aerobics or dance type shoes, such as ballet slippers and the like. Accordingly, the shoes 120R, 102L as contemplated by the present invention, although geared especially towards athletics and court-based sports, may nonetheless take any of a variety of footwear known in the art.

In combination with each respective shoe 102R, 102L is an elongate sleeve 104R, 104L operatively interconnected therewith. As illustrated, each respective elongate sleeve 104R, 104L has an upwardly-extending proximal end 104Ra, 104La that can extend as high as the upper thigh of the wearer, as shown by upwardly-extending proximal end 104Ra, but that such proximal end may be folded down as may be desired by the wearer to just above the knee, as shown by 104La, or any point between the upper thigh of the wearer down to the wearer's ankles, as may be desired. The elongate sleeve portions 104R, 104L are interconnected with dedicated ones of the pair of shoes 102R, 102L such that each respective distal end of the sleeves 104Rb, 104Lb preferably extend about the respective ankles of the wearer and into each respective shoe 102R, 102L. To maintain each respective sleeve 104R, 104L with the dedicated shoe to which it is attached 102R, 102L, it is contemplated that the distal ends of the sleeves 104Rb, 104Lb will be affixed to the shoes 102R, 102L via an attachment mechanism such as stitching 106 as shown. In this regard, it is contemplated that the respective shoes 102 and legging sleeves 104 will cooperate to form a protective and supportive covering extending over the feet and up to the upper thigh of the wearer as a continuous structure. Such interconnection, shown by 106, also enables the legging sleeves 104R, 104L to remain in proper registry and orientation relative their respective shoe 102R, 102L to which the same is attached to thus ensure that the optimal degree of support is continuously applied to the anatomical regions extending from the foot, ankles, calves/shin, knee and ultimately the thigh of the wearer, and thus resist slipping, rotational movement and the subsequent consequential discomfort and suboptimal support typically associated by simply wearing disconnected elements as discussed in the Background.

With respect to each respective supportive legging sleeves 104R, 104L, the same may be fabricated from a wide-variety of materials well-known in the art. In this regard, it is contemplated that such legging sleeves 104R, 104L may be fabricated from supportive, elastic material, such as LYCRA® and the like so as to provide sufficient anatomical support while also allowing the wearer to engage in rigorous sports activities and make sudden movements over a wide range of motion. It is further contemplated that the legging sleeves 104R, 104L may be fabricated from material that is particularly well suited to provide a wicking function to mitigate the effects of heavy sweating, as well as may be fabricated to allow for ventilation to those areas of the ankle, calf/shin, knee and thigh sought to be supported thereby. Still further, it will be readily understood by those skilled in the art that the material from which the legging sleeves 104R, 104L are fabricated may be selectively chosen or treated to impart a desired function or aesthetic effect. Along those lines, it is contemplated that materials from which the legging sleeves 104R, 104L may be fabricated may be operative to provide enhanced protection form ultraviolet rays, may be provided with reflective material to increase visibility, may be fashioned so as to have fanciful designs and art work, or include logos, branding, advertisements or the like.

To further increase the functionality of the invention 10, it is further contemplated that legging sleeves 104R, 104L may be designed to facilitate the interconnection of additional supportive or protective devices as may be desired by the wearer or to enhance protection or support when the wearer engages in a particular activity or sport. As illustrated in FIG. 1, for example, legging sleeve 104R may include a further knee pad support 108 for selectively positioning a knee pad 110 in a proper position and orientation in front of the wearer's knee. To that end, it is contemplated that such support structures as 108 may be formed as channels, grooves, pockets or other types of pouches, containers and the like that easily and readily provide means for interconnecting protective elements such as knee pad 110 in position to thus easily and readily provide an enhanced degree of protection to the wearer.

Similarly, and as also shown in FIG. 1 with respect to left legging sleeve 104L, the legging sleeve may be provided with a shin guard-type mechanism whereby a pocket 112 is formed over the wearer's shin to define a pocket 114, within which may be positioned a shin guard, well known in the art with respect to sports such as soccer and football. In this regard, it is contemplated that such protective channels, grooves, pockets and the like, such as 108 and 112, may be formed anywhere about a respective sleeve 104 as may be desired for a particular protective element suited for a particular sport or activity.

Along those lines, although not shown, it is contemplated that such channels, pockets, pouches and the like may be further specifically formed to receive and interconnect with conventional bracing for providing support to the ankle and/or knee of the wearer. In this regard, braces such as ankle braces and knee braces are well-known in the art and may be selectively deployed through the combination shoe and legging sleeve 10 of the present invention such that such braces may be attachable to either of the shoe 102 or legging sleeve 104, or both, such that the brace is maintained in a proper orientation relative the wearer's leg such that an optimal degree of support/bracing is imparted thereby while minimizing interference with the wearer's ability to perform quick movements.

Advantageously, given the continuous design extending from the footwear 102 through legging sleeve 104, protective inserts or bracing can extend from the foot to the upper thigh with stiff or moderate support as may be desired. In addition, such bracing and/or such protective inserts may be permanently stitched into position or may be operatively detachable. In all such cases, the invention 10 will specifically allow for support in three key areas, namely: a first area in the shoe 102, such as by an insert along the length of the shoe that goes up to the height of the shoe and upwardly therefrom to the legging sleeve 104 to cover the ankle; a second area is operative to receive protective elements as discussed above, such as inserts and support from the ankle to the knee area; and a third zone of protection extending upwardly from the knee to the upper thigh area of the wearer's leg.

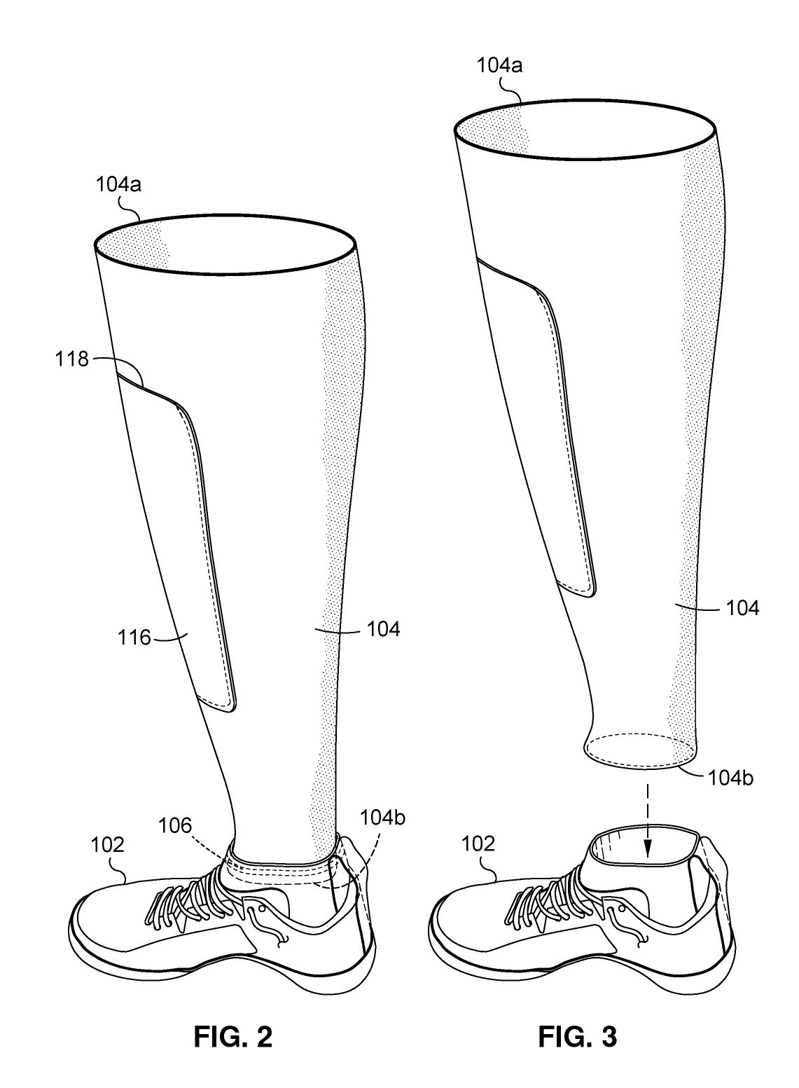

Referring now to FIGS. 2-6, and initially to FIG. 2, there are shown a variety of embodiments by which the combination shoe 102 with legging sleeve 104 of the present invention may be configured as may be desired for a particular sport or activity.

As shown in FIG. 2, the shoe 102 is interconnected to legging sleeve 104 via stitching 106 whereby the distal end 104b is stitched into the lining of shoe 102 through which the foot is received. In this regard, it is contemplated that in such embodiment, legging sleeve 104 will be permanently attached to shoe 102 such that when worn, the user will simply extend his or her foot through the opening at proximal end 104a and extend the foot, ankle, calf/shin and knee therethrough such that the proximal end 104a extends up to the thigh or anywhere from the wearer's ankle to the thigh as the wearer may desire. When worn in the manner shown, it is contemplated that any utility pouches, pockets and the like, such as 116, will thus be properly oriented and operative to interconnect or receive bracing or protective elements, such as a shin guard through pocket 118, for example.

Referring now to FIG. 3, there is shown an alternative configuration that merely shows the combination between legging sleeve 104 and shoe 102 whereby the distal end 104b of legging sleeve 104 is simply nested within shoe 102 about the ankle of the wearer. While no attachment mechanism is specified, it is contemplated that the frictional engagement between the distal end 104b and the opening of shoe 102 may in certain applications be sufficient to maintain the legging sleeve 104 in proper orientation relative shoe 102 and about the leg of the wearer.

Referring now to FIGS. 4-6, and initially to FIG. 4, alternative arrangements are shown by which the distal end 104b of legging sleeve 104 can interconnect with shoe 102 to thus form an interconnected continuum between shoe 102 and legging sleeve 104 at the juncture by which the distal end 104b interconnects with shoe 102. With respect to the embodiment shown in FIG. 4, the distal end 104b of legging sleeve 104 is provided with a band of loop material for engaging with a complementary band 122 formed on shoe 102 of hook material to thus enable the complementary bands 120, 122 to become detachably fastenable to one another. As will be appreciated by those skilled in the art, such bands 120, 122 may be selectively sewn into position and oriented so as to form a complementary interconnected fit that advantageously enables the legging sleeve 104 to be securely attached to shoe 102 in a proper orientation but also to enable the two components to be disconnected from one another for laundering purposes and the like.

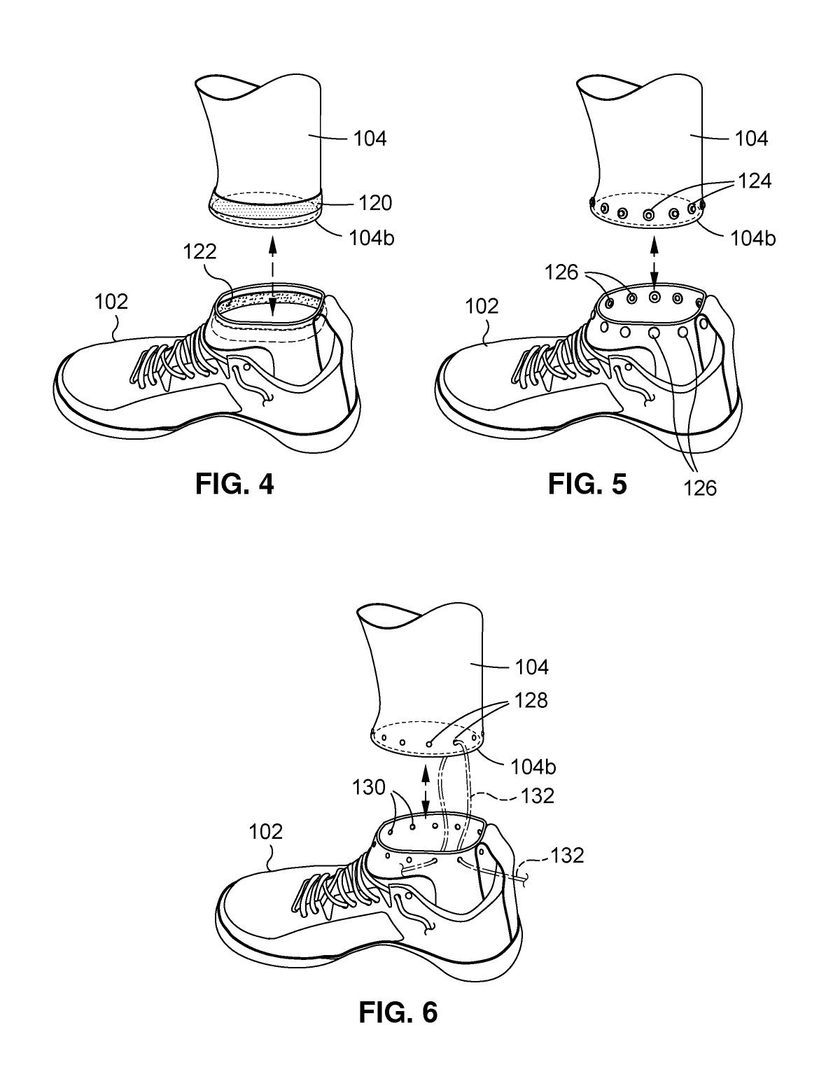

With respect to FIG. 5, there is shown an arrangement whereby a plurality of interconnectable snap fasteners 124, 126 are provided to form a interlocking relationship between the distal end 104b of legging sleeve 104 with shoe 102. As illustrated, a plurality of male fasteners 124 are formed about the distal end 104b of legging sleeve 104 that are operative to align and snap fasten with female components 126 distributed radially about the opening of shoe 102, as shown. In use, the distal end 104b will be aligned the opening of shoe 102 such that fastener components 124, 126 will be aligned with and operatively “snap” together to form a fit between shoe 102 and legging sleeve 104. Per the embodiment shown in FIG. 4, when desired, the snap fasteners may be disengaged from one another so that the legging sleeve 104 can be removed from shoe 102.

In yet a further embodiment shown in FIG. 6, the distal end 104b of legging sleeve 104 will be provided with a plurality of apertures 128 that will complement and align with a plurality of apertures 130 formed radially about the opening of shoe 102, as shown. As illustrated, a lace, cord or rope, shown in phantom as 132, may be routed through the sets of complementary apertures 128, 130 to thus enable the legging sleeve 104 to be laced, and consequently secured to shoe 102. To that end, it is contemplated that the apertures 128, 130 may be formed similar to eyelets formed on shoes and the like and that a conventional shoelace or cord may be used to lace the two together.

In further alternative embodiments that will be readily appreciated by those skilled in the art, it should be recognized that other interconnecting mechanisms, such as straps, buttons, buckles, belts or other types of mechanisms can be readily implemented to facilitate the ability of shoe 102 to interconnect with legging sleeve 104 about the distal end 104b of the legging sleeve 104. Accordingly, any such variation on attachment mechanisms will be deemed within the scope of the present invention.

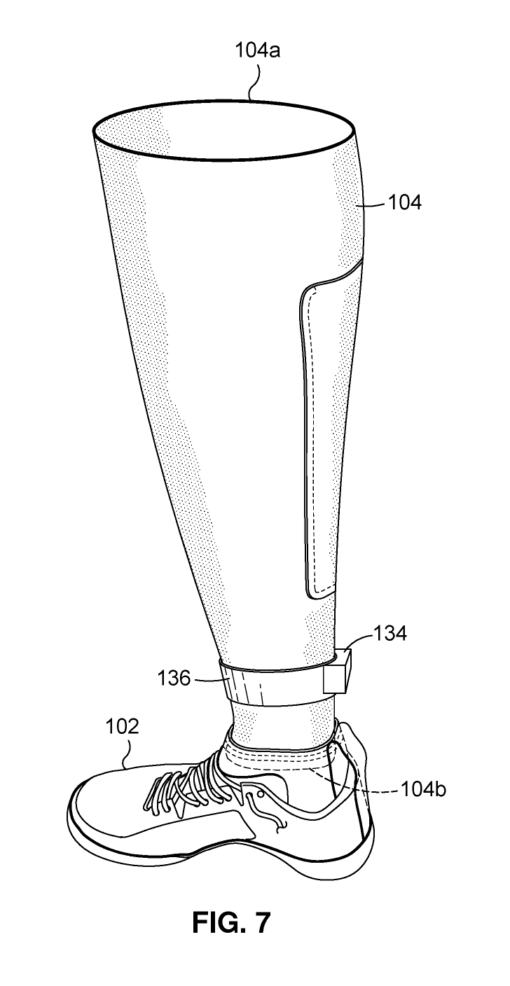

Referring now to FIG. 7, there is shown a further refinement of the present invention whereby shoe 102 with interconnected sleeve 104 further includes a sensor element 134 that is secured about legging sleeve 104 via strap 136. In such embodiment, it is contemplated that sensor 134 may take any of a variety of forms that are operative to generate and keep track of data indicative of one or more physiological parameters of the wearer and/or is able to generate data indicative of a particular measurement, time interval or other useful information that can be used in a variety of different applications. For example, such sensor component 134 may be operative to monitor the wearer's heart rate or number of calories burned, as well as other possible physiological parameters such as blood pressure and the like. Such sensor 134 may be operative to measure the distance the wearer travels, such as number of steps taken, distance ran while playing a given sporting event or activity, or measure an elapsed period of time, as may be utilized in training or rehabilitation purposes.

Along those lines, it is contemplated that the embodiment depicted in FIG. 7 may take any of a variety of forms whereby sensor 134 may be positioned anywhere about the shoe 102 and/or legging sleeve 104, and that multiple sensors may be utilized in different locations to measure different parameters or activity. In this respect, it is contemplated that the integration of sensors, such as 134, may facilitate the ability of the present invention to be utilized in a variety of applications such as physical therapy and rehabilitation, especially rehabilitation including the knees, ankles and feet, although other physical therapy applications may involve other target areas beyond the feet, ankle, knee and thigh of the user, such as the back, shoulder and neck of the wearer.

Indeed, it is contemplated that sensors such as 134 may be utilized to deploy other applications, such as use as built-in electrodes for imparting electrical stimulation or other possible electro-mechanical controls to direct and control movement, directional speed and the like, as well as measure any such movement, speed and the like and transmit that data to a receiving device. In one specific application, it is contemplated that sensors, such as 134, may comprise built-in electrodes that may be selectively positioned about the feet, ankle, calf and knee or thigh of the wearer and operative to selectively impart electrical stimulation to all or a portion of such anatomical target areas for purposes of pain relief, with frequency, amplitude and duration of the electrical stimulation being either pre-programmed or selectively imparted by the wearer or a third-party, such as a doctor or physical therapist.

Still further, it is contemplated that various combinations of hardware and/or software can be incorporated as part of the invention 10, and implemented through sensors such as 134 that allows transmission of data from such sensors, as well as for such sensors 134 to receive signals. Via the reception and transmission of such signals, it is contemplated that opportunities are created through the combination shoe/legging sleeve 10 of the present invention whereby signals received and transmitted thereby create an opportunity for developers to create their own applications for the purpose of monitoring the wearer's activities and physiological responses, and the data generated thereby manipulated via software applications for not only monitoring health and body activities, but also for other purposes such as gaming and/or device control to thus assist as part of a training or conditioning regimen for a particular sport, such as tennis and the like. For example, it is further expressly contemplated that sensors, such as 134, may be operative to respond to signals received from an external device that can in turn be integrated into a workout regimen whereby the receipt of such signal can correspond to adjusting resistance in relation to a particular activity, encourage the wearer to move in a particular manner or otherwise facilitate the wearer to perform a given movement more quickly or with greater endurance.

Sensors can also be utilized to generate data and provide means for training and improving balance, endurance and coordination, such as through the use of center-of-gravity sensors that can allow wearers of the present invention to provide directional signals by stressing a certain position during a given activity, such as the assessment of the wearer's movements during the service of a tennis ball, the service of a volleyball, the performance of a jump shot in basketball, or swing of a golf club. It is further believed that such sensors have tremendous potential for use in gaming applications, such as practicing dance moves, speed and agility competition and any of a variety of activities well-known and understood in the art.

Indeed, by virtue of the activity and motion generated by the wearer as a result of wearing the combination shoe and legging sleeve of the present invention, it is contemplated that the same may further be utilized to mechanically generate electricity in some fashion, which in turn can be used for a variety of applications, such as generating light, biofeedback mechanisms, powering one or more of the sensors, powering an electronic device plus numerous other applications that will be readily understood and appreciated by those skilled in the art.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. Footwear for providing structural support to the leg of the wearer comprising:
an athletic shoe, and
an elongate legging sleeve having a proximal and a distal end said distal end being integrally connectable with said shoe such that said sleeve is operatively positionable upon the wearer's leg whereby said proximal end fits about a portion of the thigh of said wearer and said distal end extends about said ankle of said wearer.

2. The footwear of claim 1 wherein said shoe is selected from the group consisting of a basketball shoe, tennis shoe, jogging shoe, volleyball shoe, football cleat, baseball cleat, soccer cleat, golf shoe, track shoes, hiking boot, aerobics shoe and ballet slipper.

3. The footwear of claim 2 wherein said distal end of said legging sleeve is sewn into said shoe.

4. The footwear of claim 2 wherein said distal end of said legging sleeve is interconnectable to said shoe via a fastening mechanism selected from the group consisting of hook and loop fasteners, snap fasteners, buttons, laces, buckles, straps and latches.

5. The footwear of claim 1 wherein said legging sleeve is fabricated from an elastic material and operative to provide compressive support to the thigh, calf and ankle of the wearer when worn thereabout.

6. The footwear of claim 1 wherein said legging sleeve is fabricated from a reflective material.

7. The footwear of claim 1 wherein said legging sleeve is formed from a material operative to provide protection from ultraviolet rays.

8. The footwear of claim 1 wherein said footwear further includes a brace for providing support to a portion of the wearer's leg; and
said legging sleeve further includes a sleeve or pocket for receiving and securing said support structure in position upon the wearer.

9. The footwear of claim 8 wherein said brace comprises an ankle brace.

10. The footwear of claim 8 wherein said brace comprises a knee brace.

11. The footwear of claim 1 wherein said footwear further comprises a protective covering positionable upon a portion of the wearer's leg and said legging sleeve is operative to receive and interconnect with said protective covering.

12. The footwear of claim 11 wherein said protective covering comprises a shin guard and said legging sleeve includes a pocket for receiving and holding said shin guard in position.

13. The footwear of claim 11 wherein said protective covering comprises a knee pad and said legging sleeve includes a pocket for receiving and holding said knee pad in position.

14. The footwear of claim 1 wherein said footwear further comprises a sensor mechanism interconnectable therewith for measuring the degree of physical activity of the wearer.

15. The footwear of claim 14 wherein said sensor is operative to determine the number of calories burned by the wearer, determine the distance moved by the wearer, measure an increment of time the sleeve is worn by the wearer, and measure the wearer's heartrate; and wherein said legging sleeve is operative to receive and interconnect with said sensor.

16. The footwear of claim 1 wherein said footwear further comprises a sensor mechanism interconnectable therewith for receiving signals and directing the wearer to move in a directed fashion.

17. The footwear of claim 16 wherein said sensors are operative to facilitate movement corresponding to a motion or movement selected from the group consisting of serving a tennis ball, serving a volley ball, swinging a golf club, and performing a dance move.
